(19) United States
(12) Patent Application Publication
(10) Pub. No.: US 2008/0161596 A1
(43) Pub. Date: Jul. 3, 2008

(54) Method for Preparing (Meth) Acrylic Esters or Anhydrides

(75) Inventors: Alain Riondel, Forbach (FR); Jean-Philippe Gendarme, Saint Avold (FR); Gilles Herbst, Nancy (FR)

Correspondence Address: BUCHANAN, INGERSOLL & ROONEY PC, POST OFFICE BOX 1404, ALEXANDRIA, VA, 22313-1404, US

(73) Assignee: ARKEMA FRANCE, Puteaux (FR)

(21) Appl. No.: 11/664,938

(22) PCT Filed: Oct. 11, 2005
(86) PCT No.: PCT/FR2005/002508

§ 371 Date: May 17, 2007

(30) Foreign Application Priority Data
Oct. 12, 2004 (FR) 0410734

(51) Int. Cl. C07C 69/66; B01J 8/06

(52) U.S. Cl. 560/155; 422/212; 560/129

(57) ABSTRACT

The invention concerns a method for preparing (meth)acrylic esters or anhydrides from acrylic acid of formulae (I), where R is H or Me, and R′ is alkyl and R″ is alkyl or ethyl substituted by dialkylamino whereof the alkyls can form with the nitrogen atom, a 5- or 6-membered heterocycle, capable of bearing one or several alkyl substituents. OII or ═O, or R′ is H and R″ is alkyl, the alcohol R″—OH being primary or secondary; or (II) wherein R′ is H or Me, functioning preferably in the presence of at least one catalyst, wherein is performed a reaction in a reaction R2 with constant equilibrium displacement provided with a first reactor R1 designed for prior dehydration of the reagents to be involved in the reaction.

The present invention relates to a method for preparing (meth)acrylic esters or anhydrides, either by transesterification, or by esterification from (meth)acrylic acid, in a continuous manner, in a continuous equilibrium shift reactor.

In European patent application EP 960877 the continuous preparation of dialkylaminoalkyl (meth)acrylate in the presence of a tetraalkyl titanate as transesterification catalyst and in the presence of at least one polymerization inhibitor has been described. The reaction is carried out at 90-120° C., then the crude reaction mixture is sent to a first distillation column, under reduced pressure. The distillation carried out makes it possible to obtain a flow comprising the expected ester and light products and which are substantially free of catalyst. This flow is sent to a second distillation column under reduced pressure, in which another flow is collected comprising the expected ester, traces of light products and heavy by-products as well as polymerisation inhibitor/inhibitors. Rectification is carried out in a third distillation column, under reduced pressure, in which the expected ester is isolated.

In French patent application FR 2389070 the continuous preparation of dimethylaminoethyl acrylate by reaction of dimethylaminoethanol with ethyl acrylate, in the presence of at least one transesterification catalyst, has been described. The reaction is carried out in the presence of tetraalkyl titanate and in the presence of at least one polymerization inhibitor. The reaction is carried out in several tubular plug-flow reactors, preferably placed in series. The azeotropic ethyl acrylate—ethanol mixture formed is continuously drawn off from each piston reactor, during the reaction. The residence time of the reaction is 2 to 6 hours. However the installation which consists of the placement in series of multiple tubular piston reactors, has limits from the industrial point of view due to the increase in the amount of equipment.

In the German patent application DE 10127938 the preparation of dialkylaminoethyl (meth)acrylates by esterification of (meth)acrylic acid alkyl esters in the presence of a catalyst and in which the residence time of the reaction mixture in the reaction zone is 1.5 to 3 hours, has been described.

In the German patent application DE 10127939 the preparation of (meth)acrylic esters by transesterification with an alcohol in the presence of a catalyst has been described. The alcohol can in particular be dialkylaminoethanol. The dialkylaminoethyl (meth)acrylates prepared have a good degree of purity.

Considering the above, it is clear that the industrial implementation of the reaction for the preparation of (meth)acrylic esters or anhydrides is not simple as a result of the use of one or more catalysts, the presence of at least one polymerization inhibitor, the formation of azeotropes and/or the numerous by-products formed; all these substances on the one hand must not interfere with the different stages of the reaction and on the other hand must be separated in order to shift the equilibrium of the reaction and to isolate the ester or the expected product. Moreover, it is indispensable to ensure that the deactivation of the catalyst is prevented. Until now, in numerous cases of balanced reactions, it was common to use multitubular reactors. However, this type of installation did not allow the intermediate drawing off of a reaction product or by-product. In certain cases this could lead to parasitic reactions between the product obtained and the initial reagent. For example in the patent U.S. Pat. No. 5,216,179 it has been shown that acrolein reacts with the initial glycerol, in catalytic medium. The continuous tubular reactors are constituted by pipework in which the reaction mixture circulates, the reagents are progressively converted into products from the inlet of the reactor to the outlet and a concentration gradient is established between the inlet and the outlet of the reactor. Thus it is particularly advantageous to be able to carry out the intermediate drawing off, either of a reaction by-product, or even of the expected product when undesirables reactions may occur leading to a drop in the yield.

It has now been found, and this is the subject of the present invention, that the method for preparing (meth)acrylic esters or anhydrides, either by transesterification or by esterification from (meth)acrylic acid could be carried out according to the diagrams:

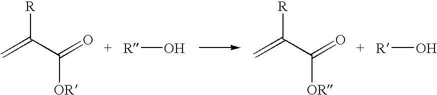

in which R represents a hydrogen atom or a methyl radical and either R′ represents a linear or branched alkyl radical containing 1 to 4 carbon atoms and R″ represents a linear or branched alkyl radical containing 4 to 40 carbon atoms or an ethyl radical substituted by a dialkylamino radical the alkyl parts of which are linear or branched and contain 1 to 4 carbon atoms or form with the nitrogen atom to which they are attached a heterocycle with 5 or 6 members, saturated, unsaturated or partially unsaturated and being able moreover to carry one or more substituents chosen from alkyl (C1 to C4), hydroxy or oxo,

or R′ represents a hydrogen atom and R″ represents a linear or branched alkyl radical containing 4 to 8 carbon atoms it being understood that the alcohol R″—OH is a primary or secondary alcohol;

or according to the diagram:

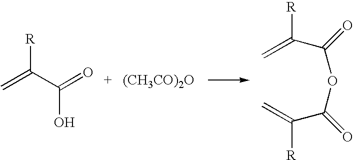

in which R represents a hydrogen atom or a methyl radical, preferably operating in the presence of at least one catalyst, in a tubular reactor R2, with continuous equilibrium shift, equipped with a first reactor R1 intended for the prior dehydration of the reagents which are to be involved in the reaction.

When the reaction for preparing (meth)acrylic esters is a transesterification reaction, the catalyst is advantageously chosen from the tetraalkyl titanates. When the reaction for preparing (meth)acrylic esters is an esterification reaction, the catalyst is advantageously chosen from acids such as for example sulphuric acid. When it relates to preparing (meth)acrylic anhydrides, the introduction of a catalyst can prove to be non-essential, nevertheless, it is also possible to operate in the presence of metal salts of anionic organic compounds having at least one carboxylic function such as for example chromium, zirconium, zinc, copper, sodium or calcium acetates or acetylacetonates.

According to the invention, it has been shown that particularly improved results of balanced reactions such as esterification or transesterification reactions or the reaction of conversion to (meth)acrylic anhydride could be obtained by operating in a reactor R2 of the plug-flow reactor type comprising a horizontal baffle-grid tubular exchanger, in which each compartment can be compared to a single perfectly stirred reactor and in which each compartment comprises in its upper part a device for drawing off gaseous products or by-products, making it possible to shift the equilibrium.

It is understood that, in certain cases, the drawing off of gaseous products, in the upper part of the compartments of the reactor R2, could also be applied to the drawing off of reaction products, in the case of balanced reactions in which the boiling points of the reagent and the reaction product/products are very different.

According to the invention, the products drawn off at the top of each compartment are treated in a distillation column which they are introduced into.

Moreover, when the catalyst used, such as for example tetraalkyl titanate, proves to be extremely sensitive to traces of water contained in the reagents and is deactivated under these conditions, it has been shown according to the present invention, that the reaction carried out in an installation constituted by a tubular reactor R2 equipped with a prior dehydration reactor R1, placed before the stage of introduction of the catalyst, made it possible to overcome this drawback.

It is understood that this installation is also comprised within the scope of the present invention.

According to a preferred mode of the invention, when it involves a transesterification reaction the catalyst used is advantageously chosen from ethyl titanate, optionally in the form of a ready-to-use solution such as a commercial solution in dimethylaminoethanol (DMAE) or in the form of a mixture with other titanates such as isopropyl titanate. When the ready-to-use catalyst is employed, the quantities used vary between 5×10⁻⁴ and 5×10⁻³ mole per mole of DMAE and preferably between 5×10⁻³ and 2×10⁻² mole per mole of DMAE.

The implementation of the methods for esterification, transesterification or preparation of acrylic anhydride in a continuous equilibrium shift reactor makes it possible to obtain a better conversion of the (meth)acrylic acid alcohol and/or acetic anhydride reagents and consequently reduced dimensions of the distillation columns for the purification stage. Moreover, the method according to the invention makes it possible to limit the residence time in the reactor and as a result to operate with a smaller reaction volume than in the methods known previously. In particular it has been shown that the residence time could be divided by 2.5 compared to a reactor, of the same dimensions, which is perfectly stirred.

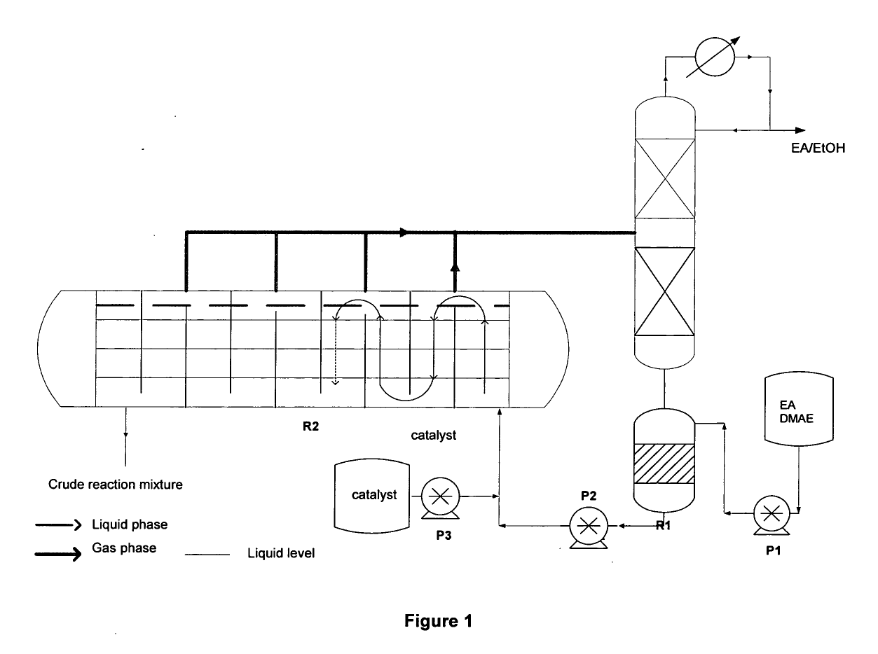

The reactors used are described in FIG. 1.

### In FIG. 1:

R1 represents the reactor into which the (meth)acrylic ester or (meth)acrylic acid, and alcohol or acetic anhydride mixture are respectively introduced. The reactor R1 is equipped with a column intended to eliminate either the azeotrope formed between the light ester and light alcohol or the azeotrope formed between the light ester and water (transesterification), or the azeotrope formed between heavy alcohol and water (esterification), or acetic acid (the case of synthesis of the anhydride). It thus makes it possible to carry out the complete dehydration of the reagents. The polymerization inhibitor is also introduced into this reactor.

R2 represents a horizontal baffle-grid tubular reactor into which the reaction mixture is sent from the outlet of the reactor R1 via the regulating pump P2. The reactor R2 also receives the catalyst via the regulating pump P3. In the reactor R2, each baffle part in its upper part can be compared to an individual reactor placed in series with the following. Advantageously, the reactor can comprise 2 to 14 baffles situated in the upper part, in such a way as to be able to be comparable with 3 to 15 reactors in series. Generally a reactor R2 comprising 8 baffles (therefore 9 compartments) is particularly efficient. At the upper level of each compartment a system is arranged for communication with the column for treatment of the products or by-products such as the azeotrope formed between (meth)acrylic ester and light alcohol, the water/heavy alcohol azeotrope or acetic acid.

The reactors R1 and R2 are heated to temperatures comprised between 100 and 160° C. Preferably to a temperature close to 140° C. Thus, in the reactor R2, from the first reactor to the last, the temperatures vary between 80 and 130° C.

P1, P2 and P3 represent regulating pumps.

The pump P1 makes it possible to introduce the reagents into the reactor R1 and to regulate the flow rate of their introduction. Advantageously the reactor R1 is filled to between ½ and ¾ of its capacity, preferably to ¾ of its capacity.

The pump P2 makes it possible to ensure the introduction of the reagents from R1 into the reactor R2 and to regulate the level of the reactor R1 to ¾ of its capacity.

The pump P3 makes it possible to introduce the catalyst into the reactor R2. Thus the catalyst is never in contact with acrylic ester and alcohol reagents in which the traces of water would not have been previously eliminated.

The crude reaction mixture can be drawn off by gravity at the level of the last compartment of the tubular reactor R2. It can be purified, according to the usual methods.

It is understood that the device comprising the reactors R1, R2 equipped with the devices for drawing off in the upper part of each compartment, and the pumps P1, P2, P3 are also comprised within the scope of the present invention. It is understood that the reactor R2 is also comprised within the scope of the present invention.

The reactions are carried out after the installation is brought on stream. The residence time is determined after the installation is brought on stream. The residence time in reactor R2 is 3 hours. After 2 residence times, the apparatus is operational. After starting the operation of the installation, advantageously 2 or 3 repetitions of residence time take place. Advantageously the operation takes place under reduced pressure between 800 and 1015 mbars and preferably between 900 and 1000 mbars.

The method according to the invention has the advantage of being easily industrialized. It makes it possible to limit the residence time in the reactor and as a result to operate with a smaller reaction volume than in the methods known previously. In particular it has been shown that the residence time could be divided by 2.5 compared to a reactor, of the same dimension, which is perfectly stirred.

Finally, the implementation of the method for (meth)acrylic ester transesterification in a continuous equilibrium shift reactor makes it possible to obtain a better conversion of the reagent: alcohol such as for example dimethylaminoethanol, and consequently reduced dimensions of the distillation columns in the purification stage. A better selectivity is also obtained.

The polymerization inhibitors can be chosen from the inhibitors which are usually used and which do not alter the molecules of the present reaction. In particular phenothiazine (PTZ), tetramethyl-4-hydroxy-1-piperidinyloxy (4-HO-TEMPO) (in the case of esterification or transesterification), tertbutylcatecol, 2-orthoditertbutylparacresol (BHT), hydroquinone methyl ether (HQME), hydroquinone and/or their mixtures are used. The polymerization inhibitor is introduced with the mixture of the reagents into the reactor R1, by the pump P1. Advantageously 100 to 5000 ppm of polymerization inhibitor is used compared to the reaction charge introduced into R1.

According to a preferred mode of the invention, the method for preparing an acrylic ester such as dimethylaminoethyl acrylate by continuous transesterification of ethyl acrylate by dimethylaminoethanol can be carried out by operating in the presence of at least one catalyst such as tetraalkyl titanate in a continuous equilibrium shift reactor R2 equipped with a first reactor R1 intended for the prior dehydration of the reagents which are to be involved in the reaction and for the elimination of the azeotrope formed between ethyl acrylate and water.

According to a preferred embodiment in FIG. 1:

R1 represents the reactor into which the ethyl acrylate (EA) and dimethylaminoethanol (DMAE) mixture is introduced. The reactor R1 is equipped with a column intended to eliminate the azeotrope formed between ethyl acrylate and water, as well as the azeotrope formed between ethyl acrylate and ethanol. It also makes it possible to carry out the complete dehydration of the reagents. The polymerization inhibitor is also introduced into this reactor.

R2 represents the horizontal baffle-grid tubular reactor, into which the reaction mixture is sent. Each compartment is equipped in its upper part with a system for elimination of the vapour phase. A communication system is arranged between the reactor R2 and the column for elimination of the ethyl acrylate—ethanol azeotrope. Said azeotrope is then injected into the distillation column, preferably halfway up the column.

The reactors R1 and R2 are heated to temperatures comprised between 100 and 160° C. Preferably to a temperature close to 140° C. Thus, in the reactor R2, from the first reactor to the last, the temperatures varies between 80 and 130° C.

P1, P2 and P3 represent the regulating pumps.

The catalyst is chosen from the tetraalkyl titanates, such as for example tetraethyl titanate in solution in DMAE.

The crude reaction mixture drawn off at the level of the last compartment of the tubular reactor R2 can be purified, according to the usual methods, in particular operating according to the method described in application EP 960877.

The following example shows how the invention can be put into practice.

### EXAMPLE

In the following example, the flow rates correspond to a “residence time” of 3 hours for 9 compartments of the reactor, under reduced pressure: 91 kPa.

A solution of ethyl acrylate (EA) and dimethylamino-ethanol (DMAE) in a molar ratio EA/DMAE=1.56 is prepared and stabilized with 2010 ppm of phenothiazine (PTZ) and 50 ppm of 4-HO TEMPO (tetramethyl-4-hydroxy-l-piperidinyloxy) by successively introducing into a 5 litre tank:

1500 g of DMAE

2628 g of EA

8.3 g of PTZ

0.21 g of 4-HO TEMPO

The catalyst used is prepared in the form of a commercial solution at 80% by weight ethyl titanate in previously prepared and ready-to-use dimethylaminoethanol.

It is used at a rate of 2.85 g/h.

The vacuum pump, the refrigeration system, the bubbling of dry air into the reactor R1, as well as heating the reactors R1 and R2 to 140° C., are set in operation. The mixture EA and DMAE (comprising PTZ and 4-HO TEMPO) is introduced into the reactor R1, with a flow rate of 278 g/h by the pump P1. When R1 is ¾ full (100 ml), R2 is fed from R1. The level of the reactor R1 is maintained constant by the presence of the accelerator pump P2 (flow rate of 694.5 g/h in a steady state).

The pump P3 for introduction of the catalyst is actuated with a flow rate of 2.85 g/h.

When the temperature at the head of the column is equal to the boiling temperature of the EA/EtOH azeotrope, the column is in a steady state (T=71° C.) and the drawing off can be carried out, with a reflux of 5 to 1 (Fraction F1). After bringing the installation on stream (6 hours), the reaction is carried out for a duration of 6 hours (2 repetitions of “residence time”), by drawing off the EA/EtOH azeotrope in the upper part of each of the 9 compartments. The crude reaction mixture is analyzed by gas chromatography in order to determine the selectivity and the yield of DAMEA (dimethylaminoethyl acrylate), as well as the conversion of DMAE.

During this test 606.5 g of crude reaction mixture is recovered and 134.5 g in total of fraction F1 comprising the EA/EtOH azeotrope.

In the reactor R2, the temperature has varied from 85° C. in the 1^{st} compartment to 120° C. in the last compartment.

### Crude Reaction Mixture:

DAMEA: 60.34%

EA: 28.24%

DMAE: 8.9%

EtOH: 2.52%

i.e. a DAMEA yield of 76% and a DMAE conversion of 82%, or a DAMEA selectivity of 92.7%.

### COMPARATIVE EXAMPLE

The operation is carried out strictly in accordance with the conditions described in the example above, but without the introduction of reactor R1 and with DMAE containing 1000 ppm of water.

At the end of the test a drop in yield of 15% is observed.

1. A method for the preparation of (meth)acrylic esters or anhydrides, 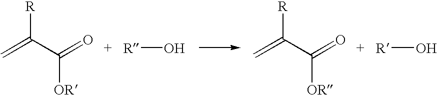
either by transesterification, or by esterification from acrylic acid, according to the diagrams: in which R represents a hydrogen atom or a methyl radical, and either R′ represents a linear or branched alkyl radical containing 1 to 4 carbon atoms and R″ represents a linear or branched alkyl radical containing 4 to 40 carbon atoms or an ethyl radical substituted by a dialkylamino radical the alkyl parts of which are linear or branched and contain 1 to 4 carbon atoms or form with the nitrogen atom to which they are attached a heterocycle with 5 or 6 members, saturated, unsaturated or partially unsaturated and being able moreover to carry one or more substituents chosen from alkyl (C1 to C4), hydroxy or oxo, or R′ represents a hydrogen atom and R″ is a linear or branched alkyl radical containing 4 to 8 carbon atoms, it being understood that the alcohol R″—OH is a primary or secondary alcohol; or according to the diagram: 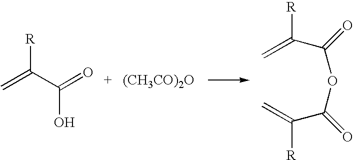
in which R represents a hydrogen atom or a methyl radical,
preferably operating in the presence of at least one catalyst, wherein the reaction is carried out in a continuous equilibrium shift reactor R2, equipped with a first reactor R1 intended for the prior dehydration of the reagents which are to be involved in the reaction.

2. A method for the preparation of (meth)acrylic esters or anhydrides according to claim 1, wherein, when the reaction is a transesterification reaction, the catalyst is chosen from the tetraalkyl titanates, when the reaction is an esterification reaction, the catalyst is chosen from acids, and when it involves the preparation of (meth)acrylic anhydrides, the introduction of a catalyst can prove to be non-essential, or the catalyst is chosen from metal salts of anionic organic compounds having at least one carboxylic function.

3. A method for preparing dimethylaminoethyl acrylate, by continuous transesterification of ethyl acrylate by dimethylaminoethanol, in the presence of at least one catalyst comprising tetraalkyl titanate, wherein the reaction is carried out in a continuous equilibrium shift reactor R2, equipped with a first reactor R1 intended for the prior dehydration of the reagents which are to be involved in the reaction.

4. A method according to claim 1, wherein the reactor R2 is a reactor of the plug-flow reactor type comprising a horizontal baffle-grid tubular exchanger.

5. A method according to claim 1, wherein the reactor R2 comprises 2 to 14 baffles situated in the upper part, forming 3 to 15 reactors in series.

6. A method according to claim 5, wherein the reactor R2 comprises 8 baffles situated in the upper part, forming 9 compartments.

7. A method according to claim 1, wherein the crude reaction mixture is drawn off by gravity at the level of the last compartment of the reactor R2.

8. A method according to claim 1, wherein a device for drawing off of gaseous products or by-products is situated in the upper part of each compartment.

9. A method according to claim 8, wherein a system for elimination of the azeotrope formed between ethyl acrylate and ethanol is arranged at the upper level of each compartment of the reactor R2.

10. A method according to claim 1, wherein the catalyst used is ethyl titanate, when it is transesterification.

11. A device for the implementation of the method according to claim 1, wherein it comprises the reactor (R1) equipped with a column intended to eliminate the azeotrope, the reactor (R2): horizontal baffle-grid tubular reactor, equipped with devices for drawing off at the upper part of each compartment, and the pumps (P1) for the introduction of the reagents into (R1), (P2) for the introduction of the reagents into (R2) and (P3) for the introduction of the catalyst into the reactor (R2).

12. A reactor (R2) intended to implement the method according to claim 1, wherein it is a plug-flow reactor comprising a horizontal baffle-grid tubular exchanger, in which each compartment can be compared to a single perfectly stirred reactor and in which each compartment comprises in its upper part a device for drawing off gaseous products or by-products.
